(19) United States
(12) Patent Application Publication
(10) Pub. No.: US 2022/0285025 A1
(43) Pub. Date: Sep. 8, 2022

(54) MEDICAL SYSTEM AND CONTROL METHOD THEREOF

(71) Applicant: HTC Corporation, Taoyuan City (TW)

(72) Inventors: Yu-Shao PENG, Taoyuan City (TW); Kai-Fu TANG, Taoyuan City (TW); Edward CHANG, Taoyuan City (TW)

(21) Appl. No.: 17/653,133

(22) Filed: Mar. 2, 2022

Related U.S. Application Data
(60) Provisional application No. 63/155,311, filed on Mar. 2, 2021.

(51) Int. Cl. G16H 50/20; G16H 70/60

(52) U.S. Cl. CPC G16H 50/20; G16H 70/60

(57) ABSTRACT

A medical system is able to provide a symptom query interpretation and/or a disease diagnosis interpretation. The medical system includes an interface and a processor. The interface is configured for receiving an input state. The processor is coupled with the interface. The processor is configured to execute a symptom checker to select a current action, from a plurality of candidate symptom queries and a plurality of candidate disease predictions, according to the input state. In response to the current action is a first symptom query, the processor is configured to execute an interpretable module interacted with the symptom checker to generate a diagnostic tree for simulating possible diagnosis paths, and generate a symptom query interpretation about the first symptom query according to the diagnostic tree.

### RELATED APPLICATIONS

This application claims priority to U.S. Provisional Application Ser. No. 63/155,311, filed Mar. 2, 2021, which is herein incorporated by reference.

### BACKGROUND

### Field of Invention

The disclosure relates to a medical system for generating symptom queries during a computer aided diagnose procedure. More particularly, the disclosure relates to an AI-based medical system capable of providing explainable descriptions about the symptom queries and/or explainable descriptions about disease predictions.

### Description of Related Art

Recently the concept of computer-aided medical system has emerged in order to facilitate diagnosis for patients. The computer-aided medical system may request patients to provide some information, and then the computer aided medical system may interact with the patients by giving some symptom queries and collecting responses to these symptom queries. When this computer aided diagnose procedure is completed, the computer aided medical system will give a diagnosis or a recommendation of the potential diseases (or a register recommendation about medical department) based on the interactions with the patients. The computer-aided medical system may aid a doctor in diagnosing, or aid a patient in consulting or self-diagnosing.

Most of the computer-aided medical system utilizes an Artificial Intelligence technology (including machine learning and/or neural network model) to predict the potential diseases or give related recommendations. However, the AI-based technology usually provides the symptom queries and the result (diagnosis or recommendation) without any explanation. Therefore, it is hard for a patient or a doctor to understand why the symptom queries are given. Without proper explanations, the patient or the doctor may be confused about the result provided by the AI-based technology.

### SUMMARY

The disclosure provides a medical system, which includes an interface and a processor. The interface is configured for receiving an input state. The processor is coupled with the interface. The processor is configured to: execute a symptom checker based on a neural network model to select a current action, from a plurality of candidate symptom queries and a plurality of candidate disease predictions, according to the input state; in response to the current action is a first symptom query, execute an interpretable module interacted with the symptom checker to generate a diagnostic tree for simulating a plurality of possible diagnosis paths, each of the possible diagnosis paths passing through the first symptom query and terminated at respective a disease prediction, the possible diagnosis paths involving a positive hypothesis of the first symptom query and a negative hypothesis of the first symptom query; and generate a symptom query interpretation about the first symptom query according to the diagnostic tree.

The disclosure provides a control method include steps of: receiving an input state; utilizing a neural network model to select a current action, from a plurality of candidate symptom queries and a plurality of candidate disease predictions, according to the input state; in response to the current action is a first symptom query, generating a diagnostic tree for simulating a plurality of possible diagnosis paths, each of the possible diagnosis paths passing through the first symptom query and terminated at respective one of the candidate disease predictions, the possible diagnosis paths involving a positive hypothesis of the first symptom query and a negative hypothesis of the first symptom query; and generating a symptom query interpretation about the first symptom query according to the diagnostic tree.

The disclosure provides a non-transitory computer-readable storage medium, storing at least one instruction program executed by a processor to perform a control method. The control method include steps of: receiving an input state; utilizing a neural network model to select a current action, from a plurality of candidate symptom queries and a plurality of candidate disease predictions, according to the input state; in response to the current action is a first symptom query, generating a diagnostic tree for simulating a plurality of possible diagnosis paths, each of the possible diagnosis paths passing through the first symptom query and terminated at respective one of the candidate disease predictions, the possible diagnosis paths involving a positive hypothesis of the first symptom query and a negative hypothesis of the first symptom query; and generating a symptom query interpretation about the first symptom query according to the diagnostic tree.

In some embodiments, the AI-based medical system in the disclosure can generate symptom query interpretations associated with the symptom queries during a diagnose procedure and generate post hoc interpretations associated with the disease prediction. During the diagnose procedure, the user can understand why the current symptom query is given according to the symptom query interpretation. When the diagnose procedure is completed and a disease prediction is given by the AI-based medical system, a post hoc interpretation about the previous symptom queries and the disease prediction.

It is to be understood that both the foregoing general description and the following detailed description are demonstrated by examples, and are intended to provide further explanation of the invention as claimed.

### BRIEF DESCRIPTION OF THE DRAWINGS

Embodiments of the invention will now be described with reference to the attached drawings in which:

FIG. 1 is a schematic diagram illustrating a medical system according to some embodiments of the disclosure.

FIG. 2 is a functional diagram illustrating the interface and the processor in FIG. 1 according to some embodiments of the disclosure.

FIG. 3 is a flow chart illustrating a control method for controlling the medical system in FIG. 1 according to some embodiments of the disclosure.

FIG. 4 is a schematic diagram illustrating that a symptom query is selected as the current action according to the input state in a demonstrational example of some embodiments.

FIG. 5 is a schematic diagram illustrating the diagnostic tree generated by the interpretable module for simulating all possible diagnosis paths started from the input state and the symptom query.

FIG. 6 is a schematic diagram illustrating that a symptom query is selected as the first simulated action after the current action according to the first simulated state in a demonstrational example of some embodiments.

FIG. 7 is a schematic diagram illustrating the input state including some previous symptom queries in the previous diagnosis path according to a demonstrational example in some embodiments.

FIG. 8 is a schematic diagram illustrating the post hoc diagnostic tree generated from the previous diagnosis path by the interpretable module.

FIG. 9 is a line chart illustrating an amount variation of the disease hypotheses considered by the neural network model before and after each of the previous symptom queries according to the post hoc diagnostic tree.

### DETAILED DESCRIPTION

Reference will now be made in detail to the present embodiments of the disclosure, examples of which are illustrated in the accompanying drawings. Wherever possible, the same reference numbers are used in the drawings and the description to refer to the same or like parts.

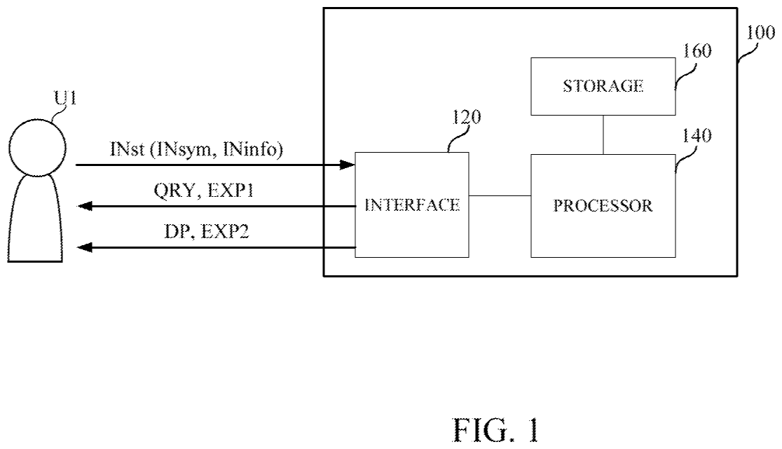

Reference is made to FIG. 1, which is a schematic diagram illustrating a medical system 100 according to some embodiments of the disclosure. As depicted in FIG. 1, the medical system 100 includes an interface 120, a processor 140 and a storage 160.

In some embodiments, the processor 140 is communicated with the interface 120. The medical system 100 is configured to interact with the user U1 through the interface 120. For example, during interacting with the user U1, the interface 120 can collect an initial symptom (as a part of an input state INst) from the user U1, provide some symptom queries QRY to the user U1, collect corresponding symptom responses (as a part of the input state INst) from the user U1. Based on aforesaid interaction history, the medical system 100 is able to analyze, diagnose or predict a potential disease occurring to the user U1, so as to generate a disease prediction DP to the user U1.

In some embodiments, the user U1 can be a patient, a family member of the patient, a friend of the patient, or a patient accompanied with a doctor. It is noticed that the medical system 100 is able to generate a symptom query interpretation EXP1 about the symptom query QRY. The symptom query interpretation EXP1 can be shown on the interface 120 along with the symptom query QRY. For example, when the interface 120 shows the symptom query QRY “do you suffer ear pain?”, and the interface 120 may also show the symptom query interpretation EXP1 “this symptom query helps to distinguish/exclude Acute Otitis Media and Flu”.

In some embodiments, the initial symptom and the symptom responses entered by the user U1 can be collected by the interface 120 as an input symptom state INsym of the input state INst.

In some embodiments, the interface 120 may further collect some other medical information INinfo (e.g., information about gender, weight, age, race, blood pressure, occupation, DNA report, test result) about the user U1 as another part of the input state INst. The medical information INinfo may also benefit to generate proper symptom queries QRY and the correct disease prediction DP. For example, if the user U1 is physical male, queries or predictions about pregnancy can be ignored.

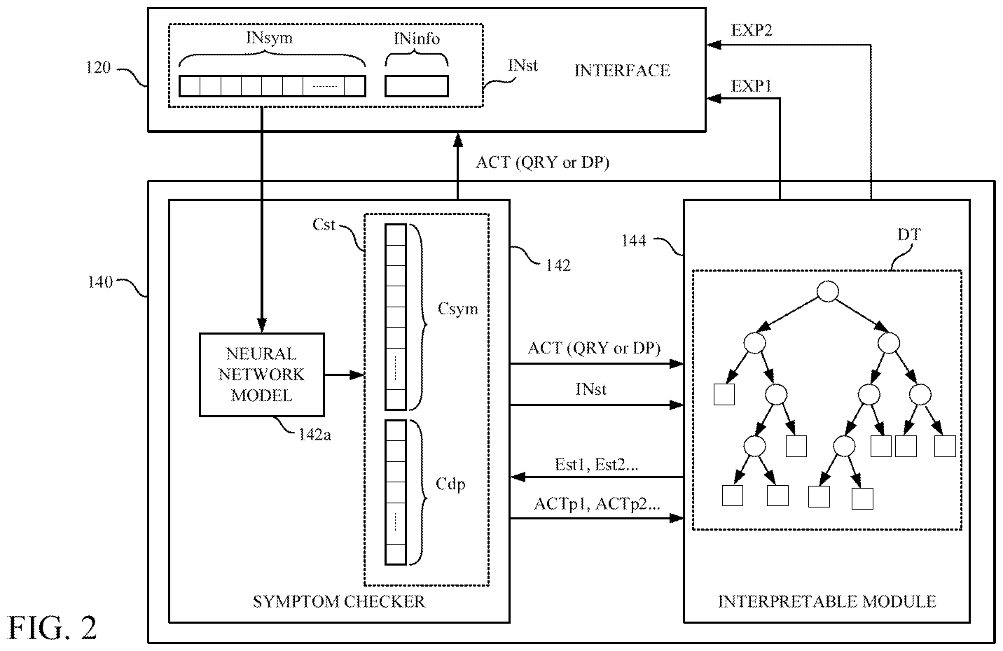

Reference is further made to FIG. 2. FIG. 2 is a functional diagram illustrating the interface 120 and the processor 140 in FIG. 1 according to some embodiments of the disclosure. As shown in FIG. 2, the processor 140 is configured to execute a symptom checker 142 and an interpretable module 144. The symptom checker 142 operates based on a neural network model 142a to select a current action ACT, from candidate symptom queries Csym and candidate disease predictions Cdp, according to the input state INst.

In some embodiments, the symptom checker 142 and the neural network model 142a are trained with a machine learning algorithm or a reinforcement learning algorithm, such that the symptom checker 142 is capable to inquire (generating the proper symptom queries QRY) and diagnose (generating the correct disease prediction DP) based on limited patient data. In some embodiments, the medical system 100 adopts a reinforcement learning (RL) framework to formulate query and diagnosis policies (e.g., Markov decision processes). In some embodiments, the symptom checker 142 and the neural network model 142a are trained based on the machine learning algorithm or the reinforcement learning algorithm by the processor 140 according to some training data (e.g., known medical records) and trained parameters about the neural network model 142a can be stored in the storage 160.

In some embodiments, the neural network model 142a is trained in advance according to some training data (e.g., known medical records). The processor 140 utilizes the neural network model 142a to generate the state value Cst and accordingly selects the sequential actions from a set of candidate actions. In some embodiments, the sequential actions include some symptom query actions, one or more medical test actions (suitable for providing extra information for predicting or diagnosing the disease) and a disease prediction action.

When the symptom checker 142 selects proper actions (e.g., some proper symptom queries, some proper medical test actions or a correct disease prediction action matching with the medical records in the training data), corresponding rewards will be provided to the neural network model 142a. In some embodiments, the neural network model 142a is trained to maximize cumulative rewards in response to the sequential actions. In some embodiments, the cumulative rewards can be calculated by a sum of a symptom abnormality reward and/or a positive/negative prediction reward. In other words, the neural network model 142a is trained to ask proper symptom queries and make the correct disease prediction at its best.

In some embodiments, the medical system 100 is established with a computer, a server or a processing center. The processor 140 can be implemented by a central processing unit (CPU), a graphic processing unit (GPU), a tensor processing unit (TPU), an application-specific integrated circuit (ASIC) or any equivalent computation unit. The interface 120 can include an output interface (e.g., a display panel for display information) and an input device (e.g., a touch panel, a keyboard, a microphone, a scanner or a flash memory reader) for user to type text commands, to give voice commands or to upload some related data (e.g., images, medical records, or personal examination reports). As shown in FIG. 1, the storage 160 is coupled with the processor 140. In some embodiments, the storage unit 160 can be implemented by a memory, a flash memory, a ROM, a hard drive or any equivalent storage component.

As shown in FIG. 1 and FIG. 2, the interface 120 can be manipulated by a user U1. The user U1 can see the information displayed on the interface 120 and the user U1 can enter his/her inputs on the interface 120. In an embodiment, the interface 120 will display a notification to ask the user U1 about his/her symptoms. The interface 120 is configured for collecting an input symptom state INsym about the user U1. The interface 120 may also collect other medical information INinfo about the user U1. The interface 120 transmits the symptom input state INst (including the input symptom state INsym and the medical information INinfo) to the symptom checker 142 of the processor 140.

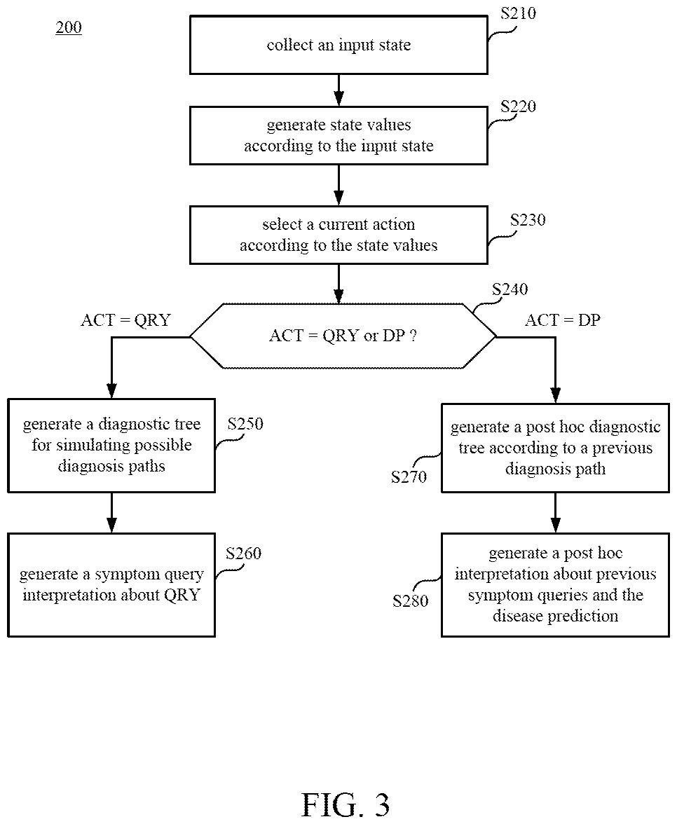

Reference is further made to FIG. 3. FIG. 3 is a flow chart illustrating a control method 200 for controlling the medical system 100 in FIG. 1 according to some embodiments of the disclosure.

As shown in FIG. 1, FIG. 2 and FIG. 3, in step S210, the interfaces 120 can collects the input state INst (including the input symptom state INsym and the medical information INinfo), and transmits the input state INst to the processor 140.

In step S220, the processor 140 receives the input state INst, and the symptom checker 142 of the processor 140 utilizes the neural network model 142a to generate current state values Cst about each of the candidate symptom queries Csym and candidate disease predictions Cdp according to the the input state INst.

In some embodiments, the neural network model 142a can be trained with a machine learning algorithm or a reinforcement learning algorithm according to the training data in advance. In some embodiments, the training data includes known medical records. The medical system 100 utilizes the known medical records in the training data to train the neural network model 142a. In an example, the training data can be obtained from data and statistics information from the Centers for Disease Control and Prevention (www.cdc.gov/datastatistics/index.html).

After training, the neural network model 142a is able to generate the state values Cst based on contents of the input state INst (including the input symptom state INsym and the medical information INinfo).

The current state values Cst are evaluated and calculated by the neural network model 142a according to the input state INst. Due to the function of the neural network model 142a, if the input state INst includes insufficient information for predicting a disease (e.g., only including two answers about symptoms without conclusive proof to predict), some state values of the candidate symptom queries Csym tend to be higher and other state values for the candidate disease predictions Cdp tend to be lower. On the other hand, if the input state INst includes enough information for predicting a disease, some state values of the candidate symptom queries Csym tend to be lower and other state values for the candidate disease predictions Cdp tend to be higher.

In step S230, the symptom checker 142 selects a current action ACT according to a maximum of the current state values Cst of the candidate symptom queries Csym and candidate disease predictions Cdp.

For example, if one of the candidate symptom queries Csym has the maximum state value, the corresponding symptom query QRY will be selected as the current action ACT. On the other hand, if one of the candidate disease predictions Cdp has the maximum state value, the corresponding disease prediction DP will be selected as the current action ACT.

In step S240, the processor 140 determines whether the current action ACT is a symptom query QRY or a disease prediction DP. If the current action ACT is the symptom query QRY (which means that the input symptom state INsym of the input state INst is not enough to make the disease prediction DP at current stage), steps S250 and S260 are executed to generate a symptom query interpretation EXP1 about the symptom query QRY.

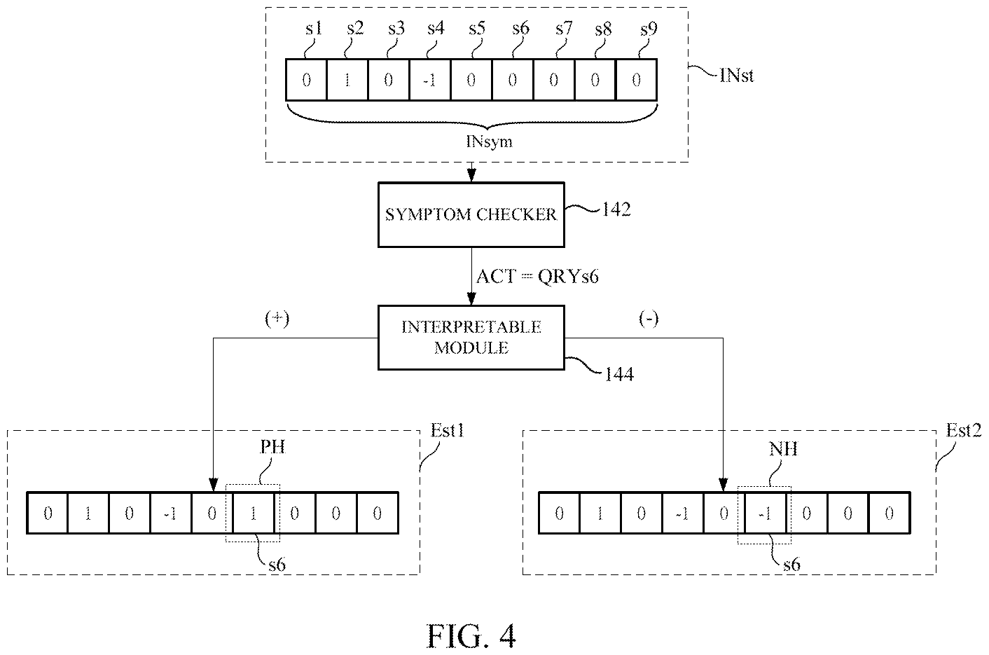

Reference is further made to FIG. 4, which is a schematic diagram illustrating that a symptom query QRYs6 is selected as the current action ACT according to the input state INst in a demonstrational example of some embodiments. In the demonstrational example shown in FIG. 4, it is assumed that the input symptom state INsym includes nine data digits s1˜s9 corresponding nine different symptom queries. Each of the data bits s1 to s9 indicates whether the user U1 has one corresponding symptom. For example, the data digit s2 is set to “1” indicating that the user U1 has a symptom “cough”; the data digit s4 is set to “−1” indicating that the user U1 does not has another symptom “headache”. The other data digits s1, s3 and s5 to s9 is set to “0” indicating that it is current unknown whether the user U1 has corresponding symptoms (e.g., “stomach pain”, “no appetite”, “fever”, “ear pain”, “shortness of breath”, . . . ) or not.

The input symptom state INsym in the input state INst only includes two confirmed answers, the data digits s2 and s4, among all symptom data digits s1˜s9. In this case, the symptom checker 142 selects the symptom query QRYs6 as the current action ACT. For example, the symptom query QRYs6 can be “do you suffer ear pain?” In some embodiments, the symptom query QRYs6 will be displayed on the interface 120.

At the same time, the symptom query QRYs6 and the input state INst are transmitted to the interpretable module 144. In step S250, the interpretable module 144 is able to interact with the symptom checker 142, and the interpretable module 144 is configured to generate a diagnostic tree DT for simulating possible diagnosis paths started from the input state INst and the symptom query QRYs6.

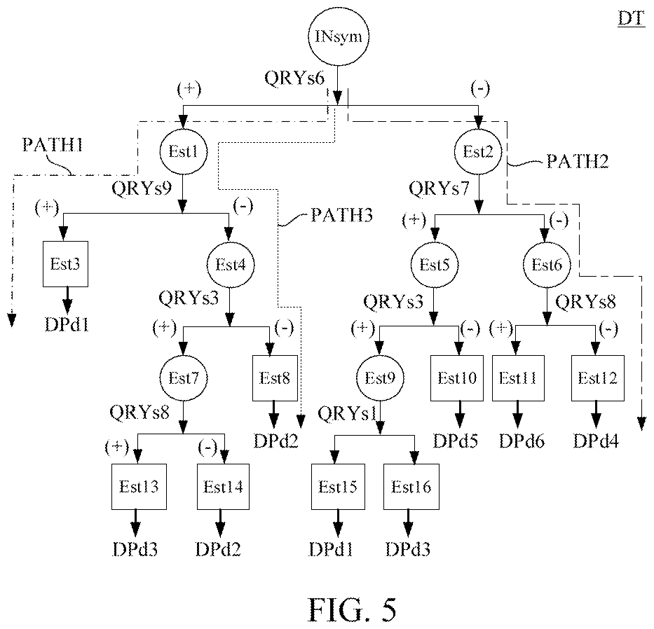

Reference is further made to FIG. 5, which is a schematic diagram illustrating the diagnostic tree DT generated by the interpretable module 144 for simulating all possible diagnosis paths started from the input state INst and the symptom query QRYs6. Details of the step S250 about how to generate the diagnostic tree DT as shown in FIG. 5 will be discussed in following paragraphs.

As shown in FIG. 4, the interpretable module 144 generates a positive hypothesis PH of the symptom query QRYs6 and fills the positive hypothesis PH into the data digit s6 relative to an input symptom state INsym in a first simulated state Est1. In other words, the first simulated state Est1 include the positive hypothesis PH “1” replacing the original data digit s6 “0” in the original input symptom state INsym in the input state INst, and all other data digits (the data digits s1-s5 and s7-s9) are duplicated from the input state INst. The first simulated state Est1 is able to simulate a situation that the user U1 enters a positive response that he/she has the symptom corresponding to the symptom query QRYs6.

As shown in FIG. 4, the interpretable module 144 generates a negative hypothesis NH of the symptom query QRYs6 and fills the negative hypothesis NH into the data digit s6 relative to an input symptom state INsym in a second simulated state Est2. In other words, the second simulated state Est2 include the negative hypothesis NH “−1” replacing the original data digit s6 “0” in the original input symptom state INsym in the input state INst, and all other data digits (the data digits s1-s5 and s7-s9) are duplicated from the input state INst. The second simulated state Est2 is able to simulate a situation that the user U1 enters a negative response that he/she does not suffer the symptom corresponding to the symptom query QRYs6.

As shown in FIG. 5, the diagnostic tree DT diverges into at least two possible diagnosis paths corresponding to the positive hypothesis PH of the symptom query QRYs6 and the negative hypothesis NH of the symptom query QRYs6. As show in FIG. 5, the possible diagnosis paths at least include a first possible diagnosis path PATH1 and a second possible diagnosis path PATH2. The first possible diagnosis path PATH1 involves the positive hypothesis PH of the symptom query QRYs6. The second possible diagnosis path PATH2 involves the negative hypothesis NH of the symptom query QRYs6.

It is noticed that the input state INst (and also the first simulated state Est1) in FIG. 4 may further include the aforementioned medical information INinfo as shown in FIG. 2. For brevity, the medical information INinfo is not illustrated in FIG. 4.

As shown in FIG. 2 and FIG. 5, the interpretable module 144 inputs the first simulated state Est1 to the symptom checker 142. The symptom checker 142 based on the neural network model 142a is able to select a first simulated action ACTp1 next to the current action ACT (the symptom query QRYs6).

If the first simulated action ACTp1 according to the first simulated state Est1 is one of the candidate disease predictions (e.g., disease predictions DPd1˜DPd6), one possible diagnosis path finishes here.

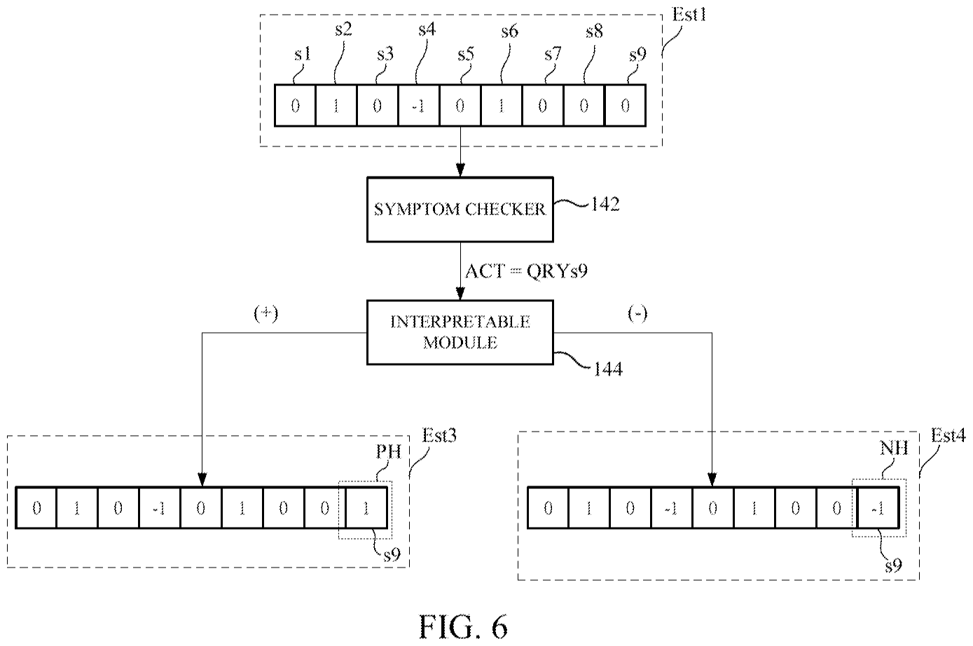

In this case, as shown in FIG. 5, the first simulated action ACTp1 according to the first simulated state Est1 is not one of the disease predictions DP, and the first simulated action ACTp1 is another symptom query QRYs9 about the data digit s9. Reference is further made to FIG. 6, which is a schematic diagram illustrating that a symptom query QRYs9 is selected as the first simulated action ACTp1 after the current action ACT according to the first simulated state Est1 in a demonstrational example of some embodiments.

As shown in FIG. 6, the interpretable module 144 generates a positive hypothesis PH of the symptom query QRYs9 and fills the positive hypothesis PH into the data digit s9 in a third simulated state Est3. In other words, the third simulated state Est3 include the positive hypothesis PH “1” replacing the original data digit s9 “0” in the first simulated state Est1.

As shown in FIG. 6, the interpretable module 144 generates a negative hypothesis NH of the symptom query QRYs9 and fills the negative hypothesis NH into the data digit s9 in a fourth simulated state Est4. In other words, the fourth simulated state Est4 include the negative hypothesis NH “−1” replacing the original data digit s9 “0” in the first simulated state Est1.

As shown in FIG. 5, the diagnostic tree DT diverges again at the symptom query QRYs9 into at least two possible diagnosis paths corresponding to the positive hypothesis PH of the symptom query QRYs9 and the negative hypothesis NH of the symptom query QRYs9. As show in FIG. 5, the possible diagnosis paths at least include the first possible diagnosis path PATH1 and a third possible diagnosis path PATH3. The first possible diagnosis path PATH1 involves the positive hypothesis PH of the symptom query QRYs9. The third possible diagnosis path PATH3 involves the negative hypothesis NH of the symptom query QRYs9.

Similarly, the interpretable module 144 can input the third simulated state Est3 to the symptom checker 142 again. The symptom checker 142 based on the neural network model 142a is able to select another simulated action next to the symptom query QRYs9. It is assumed that the symptom checker 142 select the disease prediction DPd1 next to the symptom query QRYs9. In this case, the possible diagnosis path PATH1 finished here at the disease prediction DPd1. As shown in FIG. 5, the possible diagnosis path PATH1 starts form the input state INst passing through the symptom queries QRYs6 (with the positive hypothesis) and QRYs9 (with the positive hypothesis) and terminated at the disease prediction DPd1.

As shown in FIG. 5, the interpretable module 144 can input the fourth simulated state Est4 to the symptom checker 142 again, and repeats aforesaid procedure until each of the possible diagnosis paths reaches one disease prediction. As shown in FIG. 5, the possible diagnosis path PATH3 starts form the input state INst passing through the symptom queries QRYs6 (with the positive hypothesis), QRYs9 (with the negative hypothesis) and QRYs3 (with the negative hypothesis) and terminated at a disease prediction DPd2.

Similarly, the interpretable module 144 can input the second simulated state Est2 to the symptom checker 142 again for generating a second simulated action ACTp2 according to the second simulated state Est2 as shown in FIG. 2 and generating all possible diagnosis paths under the second simulated state Est2. As shown in FIG. 5, the possible diagnosis path PATH2 starts form the input state INst passing through the symptom queries QRYs6 (with the negative hypothesis), QRYs7 (with the negative hypothesis) and QRYs8 (with the negative hypothesis) and terminated at a disease prediction DPd4.

Based on aforesaid embodiments, the interpretable module 144 can interact with the symptom checker 142 to generate the whole diagnostic tree DT as shown in FIG. 5. In some embodiments, the diagnostic tree DT includes all possible diagnosis paths diverged at every branch points (i.e., every symptom queries QRY).

After the diagnostic tree DT is generated as shown in FIG. 5, in step S260, the interpretable module 144 generate a symptom query interpretation EXP1 about the symptom query QRYs6 (i.e., the current action ACT) according to the diagnostic tree DT. In some embodiments, the symptom query interpretation EXP1 indicates two disease sets that the symptom query QRYs6 is capable to exclude or distinguish.

In some embodiments, these two disease sets are generated by comparing a set H_{O} of disease predictions under the positive hypothesis of the symptom query QRYs6 with another set H_{X} of disease predictions under the negative hypothesis of the symptom query QRYs6. As embodiments shown in FIG. 5, the set H_{O} includes the disease predictions DPd1, DPd2 and DPd3; the set H_{X} includes the disease predictions DPd1, DPd3, DPd4, DPd5 and DPd6.

In this case, the symptom query QRYs6 is able to distinguish or exclude the diseases in a union of a set H_{O}\H_{X} (which means in the set H_{O} but not in the set H_{X}) and another set H_{X}\H_{O} (which means in the set H_{X} but not in the set H_{O}).

In this demonstrational example, the set H_{O}\H_{X} includes the disease prediction DPd2; the set H_{X}\H_{O} includes the disease prediction DPd4, DPd5 and DPd6. In this case, the symptom query interpretation EXP1 indicates two disease sets, including the set H_{O}\H_{X} {DPd2} and the set H_{X}\H_{O} {DPd4, DPd5, DPd6}, indicating the disease predictions relative to the symptom query QRYs6. According to the symptom query interpretation EXP1, the user U1 can acknowledge that the symptom query QRYs6 is helpful to exclude possibilities of the disease predictions DPd4, PDd5 and DPd6 (if an answer is “YES” to the symptom query QRYs6) or to exclude a possibility of the disease prediction DPd2 (if an answer is “NO” to the symptom query QRYs6).

In this case, when the symptom query QRYs6 is displayed on the interface 120, the symptom query interpretation EXP1 about the symptom query QRYs6 can be displayed on the interface 120 along with the symptom query QRYs6.

The following Table 1 is an example about the symptom query QRYs6 and the symptom query interpretation EXP1 shown on the interface 120.

TABLE 1

| Do you suffer ear pain? | | Helpful to distinguish/exclude diseases: |
|---|---|---|
| (QRYs6) | | {DPd2} or {DPd4, DPd5, DPd6} |
| Yes | No | (EXP1) |

As shown in aforesaid embodiments, the interpretable module 144 is able to generate the symptom query interpretation EXP1 about the current action ACT (the symptom query QRYs6), such that the user U1 can realize or understand why the symptom query QRYs6 is mentioned and why this query is important. In this case, the user U1 may be more conformable in interacting with the medical system 100 and build up more trusts into the medical system 100.

Reference is made to FIG. 3 again, when the current action ACT is a disease prediction DP (which means that the input symptom state INsym of the input state INst is now enough for making the disease prediction DP at current stage) of a complete diagnose procedure, steps S270 and S280 are executed to generate a post hoc interpretation EXP2.

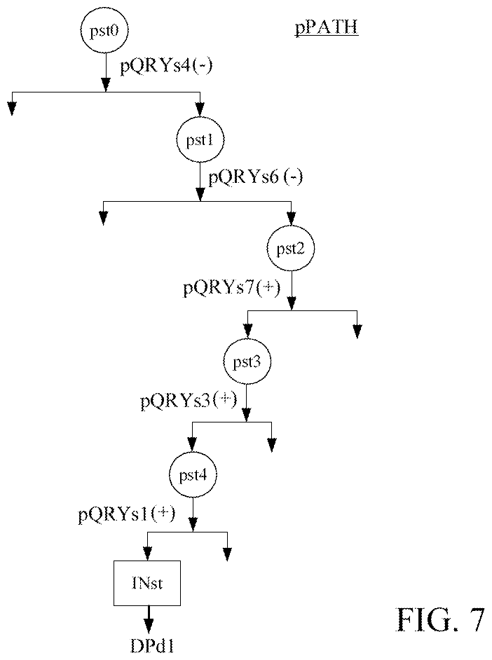

When the current action ACT is the disease prediction DP, the input state INst in this case may include symptom responses relative to previous symptom queries in a previous diagnosis path. The post hoc interpretation EXP2 is able to support why the disease prediction DP is selected, and also able to describe individual importance score about each of the previous symptom queries. Reference is further made to FIG. 7, which is a schematic diagram illustrating the input state INst including some previous symptom queries in the previous diagnosis path pPATH according to a demonstrational example in some embodiments.

In the demonstrational example, the disease prediction DPd1 is selected as the current action ACT according to the input state INst. As shown in FIG. 7, the previous diagnosis path pPATH before reaching the input state INst and the current action ACT (i.e., the disease prediction DPd1) starts from a previous state pst0 and passes through previous symptom queries pQRYs4, pQRYs6, pQRYs7, pQRYs3 and pQRYs1.

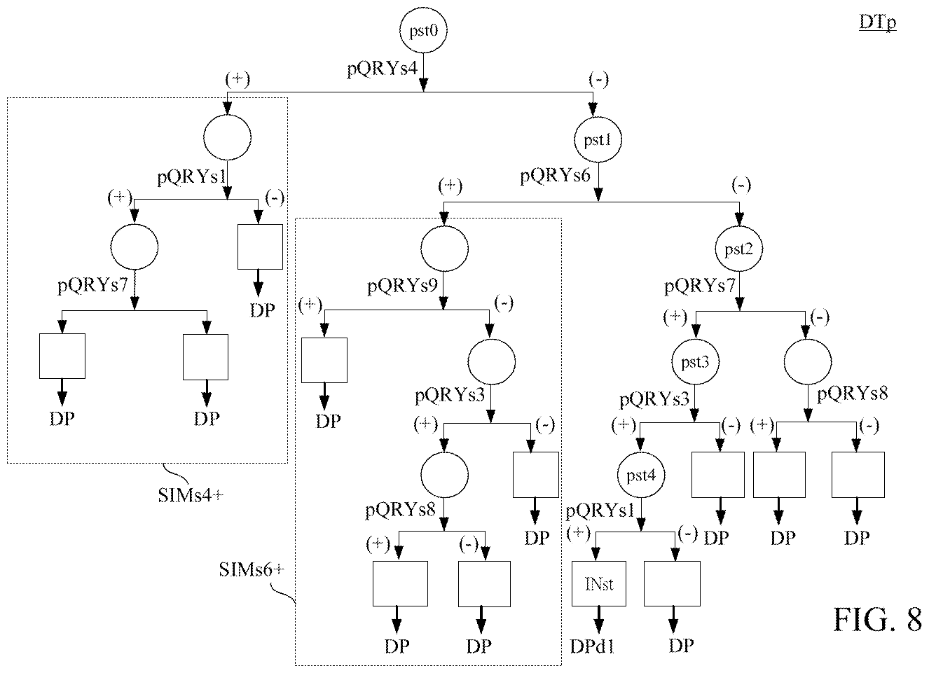

In step S270, the interpretable module 144 (interacted with the symptom checker 142) is configured to generate a post hoc diagnostic tree according to the previous diagnosis path pPATH. Reference is further made to FIG. 8, which is a schematic diagram illustrating the post hoc diagnostic tree DTp generated from the previous diagnosis path pPATH by the interpretable module 144. The post hoc diagnostic tree DTp is generated from the previous diagnosis path pPATH by simulating all possible diagnosis paths diverged at the previous symptom queries pQRYs4, pQRYs6, pQRYs7, pQRYs3 and pQRYs1 existed in the previous diagnosis path pPATH.

The details about simulating all possible diagnosis paths diverged at the previous symptom queries pQRYs4, pQRYs6, pQRYs7, pQRYs3 and pQRYs1 in the post hoc diagnostic tree DTp as shown in FIG. 8 are similar to the embodiments about simulating all possible diagnosis paths in the diagnostic tree DT as shown in FIG. 5. For example, a positive hypothesis of the previous symptom queries pQRYs4 is generated and feedback to the symptom checker 142 to simulate/calculate a portion SIMs4+ of the post hoc diagnostic tree DTp. Similarly, another positive hypothesis of the previous symptom queries pQRYs6 is generated and feedback to the symptom checker 142 to simulate/calculate another portion SIMs6+ of the post hoc diagnostic tree DTp. Other portions of the post hoc diagnostic tree DTp can be simulated and generated in the same way.

After the post hoc diagnostic tree DTp as shown in FIG. 8 is generated, in step S280, the interpretable module 144 is configured to generate a post hoc interpretation EXP2 about the previous symptom queries pQRYs4, pQRYs6, pQRYs7, pQRYs3 and pQRYs1 and the disease prediction DPd1 according to the post hoc diagnostic tree DTp.

In some embodiments, the post hoc interpretation EXP2 is generated by calculating importance scores relative to the previous symptom queries pQRYs4, pQRYs6, pQRYs7, pQRYs3 and pQRYs1.

The interpretable module 144 calculates an amount variation of the disease hypotheses considered by the neural network model 142a before and after each of the previous symptom queries pQRYs4, pQRYs6, pQRYs7, pQRYs3 and pQRYs1 according to the post hoc diagnostic tree DTp.

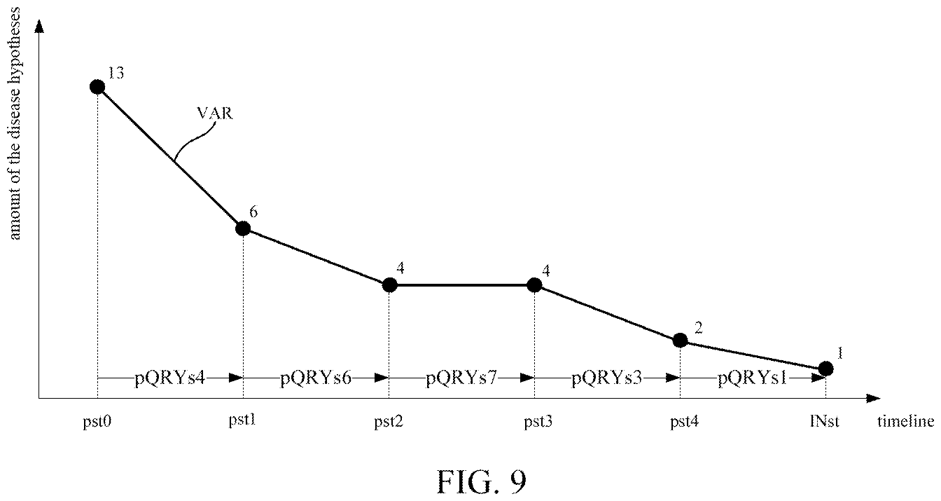

Reference is further made to FIG. 9, which is a line chart illustrating an amount variation VAR of the disease hypotheses considered by the neural network model 142a before and after each of the previous symptom queries pQRYs4, pQRYs6, pQRYs7, pQRYs3 and pQRYs1 according to the post hoc diagnostic tree DTp.

Afterward, the interpretable module 144 calculates importance scores relative to the previous symptom queries pQRYs4, pQRYs6, pQRYs7, pQRYs3 and pQRYs1 respectively according to the amount variation VAR.

The importance scores relative to the previous symptom queries pQRYs4, pQRYs6, pQRYs7, pQRYs3 and pQRYs1 can be calculated as:

$$\frac{\vert {H}_{Sk - 1} \backslash {H}_{Sk - 1} \vert}{{\sum }_{j = 1}^{t} \vert {H}_{Sk - 1} \backslash {H}_{Sk - 1} \vert}$$

The importance scores of these previous symptom queries can provide information about which one of the symptom queries is more important in giving out the disease prediction DPd1. In other words, the user U1 can acknowledge why the disease prediction DPd1 is supported by these answers to the previous symptom queries pQRYs4, pQRYs6, pQRYs7, pQRYs3 and pQRYs1 through the importance scores.

In aforesaid formula, |H_{Sk-1}\H_{Sk-1}| means the disease hypotheses considered by the neural network model 142a before and after the previous symptom query; Σⱼ₌₁^{t}|H_{Sk-1}\H_{Sk-1}| means the disease hypotheses variations considered by the neural network model 142a during the previous diagnosis path pPATH in the complete diagnose procedure.

For example, the importance score of the previous symptom query pQRYs4 can be calculated as:

$$\frac{\vert {H}_{Sk - 1} \backslash {H}_{Sk - 1} \vert}{{\sum }_{j = 1}^{t} \vert {H}_{Sk - 1} \backslash {H}_{Sk - 1} \vert} = \frac{7}{7 + 2 + 0 + 2 + 1} = \frac{7}{12} ≅ 58.33 \%$$

For example, the importance score of the previous symptom query pQRYs6 can be calculated as:

$$\frac{\vert {H}_{Sk - 1} \backslash {H}_{Sk - 1} \vert}{{\sum }_{j = 1}^{t} \vert {H}_{Sk - 1} \backslash {H}_{Sk - 1} \vert} = \frac{2}{7 + 2 + 0 + 2 + 1} = \frac{2}{12} ≅ 16.67 \%$$

After calculating the importance scores of the previous symptom queries pQRYs4, pQRYs6, pQRYs7, pQRYs3 and pQRYs1, these previous symptom queries can be shown in the post hoc interpretation EXP2 in a ranking according their importance scores. For example, the previous symptom query pQRYs4 can be labeled as the most important; and the previous symptom queries pQRYs6 and pQRYs3 can be labeled with a lower importance second to the previous symptom query pQRYs4.

Based on the importance scores of the previous symptom queries pQRYs4, pQRYs6, pQRYs7, pQRYs3 and pQRYs1 in the post hoc interpretation EXP2, the user U1 can understand more about the reasons why the previous symptom queries pQRYs4, pQRYs6, pQRYs7, pQRYs3 and pQRYs1 are mentioned and how important about the previous symptom queries pQRYs4, pQRYs6, pQRYs7, pQRYs3 and pQRYs1 in deciding the disease prediction DP. In this case, the user U1 may have more confidence in the disease prediction DP.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims.

What is claimed is:

1. A medical system, comprising:
an interface, configured for receiving an input state; and
a processor coupled with the interface, wherein the processor is configured to:
execute a symptom checker based on a neural network model to select a current action, from a plurality of candidate symptom queries and a plurality of candidate disease predictions, according to the input state;
in response to the current action is a first symptom query, execute an interpretable module interacted with the symptom checker to generate a diagnostic tree for simulating a plurality of possible diagnosis paths, each of the possible diagnosis paths passing through the first symptom query and terminated at respective a disease prediction, the possible diagnosis paths involving a positive hypothesis of the first symptom query and a negative hypothesis of the first symptom query; and
generate a symptom query interpretation about the first symptom query according to the diagnostic tree.

2. The medical system as claimed in claim 1, wherein the symptom checker based on the neural network model is configured to generate a plurality of current state values of the candidate symptom queries and the candidate disease predictions according to the input state, the symptom checker is configured to select the current action according to a maximum of the current state values.

3. The medical system as claimed in claim 1, wherein the interpretable module generates the diagnostic tree by:
generating a first simulated state comprising the positive hypothesis of the first symptom query and the input state;
inputting the first simulated state to the symptom checker to select a first simulated action next to the current action; and
in response to the first simulated action is a disease prediction, finishing one possible diagnosis path.

4. The medical system as claimed in claim 3, wherein the interpretable module generates the diagnostic tree further by:
in response to the first simulated action is a second symptom query, generating a positive hypothesis of the second symptom query and a negative hypothesis of the second symptom query; and
diverging into at least two possible diagnosis paths corresponding to the positive hypothesis of the second symptom query and the negative hypothesis of the second symptom query.

5. The medical system as claimed in claim 1, wherein the interpretable module generates the diagnostic tree by:
generating a second simulated state comprising the negative hypothesis of the first symptom query and the input state;
inputting the second simulated state to the symptom checker to select a second simulated action next to the current action; and
in response to the second simulated action is a disease prediction, finishing one possible diagnosis path.

6. The medical system as claimed in claim 5, wherein the interpretable module generates the diagnostic tree further by:
in response to the second simulated action is a third symptom query, generating a positive hypothesis of the third symptom query and a negative hypothesis of the third symptom query; and
diverging into at least two possible diagnosis paths corresponding to the positive hypothesis of the third symptom query and the negative hypothesis of the third symptom query.

7. The medical system as claimed in claim 1, wherein the symptom query interpretation about the first symptom query indicates two disease sets that the first symptom query is capable to exclude or distinguish.

8. The medical system as claimed in claim 1, wherein the input state comprises a plurality of symptom responses relative to a plurality of previous symptom queries in a previous diagnosis path, the processor is further configured to:
in response to the current action is a disease prediction of a complete diagnose procedure, execute the interpretable module interacted with the symptom checker to generate a post hoc diagnostic tree according to the previous diagnosis path, wherein the post hoc diagnostic tree comprises all possible diagnosis paths diverged at the previous symptom queries; and
generate a post hoc interpretation about the previous symptom queries and the disease prediction according to the post hoc diagnostic tree.

9. The medical system as claimed in claim 8, wherein the interpretable module generates the post hoc interpretation by:
calculate an amount variation of disease hypotheses considered by the neural network model before and after each of the previous symptom queries according to the post hoc diagnostic tree; and
calculate a plurality of importance scores relative to the previous symptom queries respectively according to the amount variation.

10. A control method, comprising:
receiving an input state;
utilizing a neural network model to select a current action, from a plurality of candidate symptom queries and a plurality of candidate disease predictions, according to the input state;
in response to the current action is a first symptom query, generating a diagnostic tree for simulating a plurality of possible diagnosis paths, each of the possible diagnosis paths passing through the first symptom query and terminated at respective one of the candidate disease predictions, the possible diagnosis paths involving a positive hypothesis of the first symptom query and a negative hypothesis of the first symptom query; and
generating a symptom query interpretation about the first symptom query according to the diagnostic tree.

11. The control method as claimed in claim 10, wherein the step of utilizing the neural network model to select the current action comprises:
generating a plurality of state values of candidate symptom queries and candidate disease predictions according to the input state by the neural network model; and
selecting the current action according to a maximum of the state values.

12. The control method as claimed in claim 10, wherein the step of generating the diagnostic tree comprises:
generating a first simulated state comprising the positive hypothesis of the first symptom query and the input state;
utilizing the neural network model to select a first simulated action next to the current action according to the first simulated state; and
in response to the first simulated action is one of the candidate disease predictions, finishing one possible diagnosis path.

13. The control method as claimed in claim 12, wherein the step of generating the diagnostic tree further comprises:
in response to the first simulated action is a second symptom query, generating a positive hypothesis of the second symptom query and a negative hypothesis of the second symptom query; and
diverging into at least two possible diagnosis paths corresponding to the positive hypothesis of the second symptom query and the negative hypothesis of the second symptom query.

14. The control method as claimed in claim 10, wherein the step of generating the diagnostic tree comprises:
generating a second simulated state comprising the negative hypothesis of the first symptom query and the input state;
utilizing the neural network model to select a second simulated action next to the current action according to the second simulated state; and
in response to the second simulated action is one of the candidate disease predictions, finishing one possible diagnosis path.

15. The control method as claimed in claim 14, wherein the step of generating the diagnostic tree further comprises:
in response to the second simulated action is a third symptom query, generating a positive hypothesis of the third symptom query and a negative hypothesis of the third symptom query; and
diverging into at least two possible diagnosis paths corresponding to the positive hypothesis of the third symptom query and the negative hypothesis of the third symptom query.

16. The control method as claimed in claim 10, wherein the symptom query interpretation about the first symptom query indicates two disease sets that the first symptom query is capable to exclude or distinguish.

17. The control method as claimed in claim 10, wherein the input state comprises a plurality of symptom responses relative to a plurality of previous symptom queries, the control method further comprises:
in response to the current action is a disease prediction of a complete diagnose procedure, generating a post hoc diagnostic tree comprising all possible diagnosis paths diverged at the previous symptom queries; and
generating a post hoc interpretation about the previous symptom queries and the disease prediction according to the post hoc diagnostic tree.

18. The control method as claimed in claim 17, wherein the step of generating the post hoc interpretation comprises:
calculate an amount variation of the disease hypotheses considered by the neural network model before and after each of the previous symptom queries according to the post hoc diagnostic tree; and
calculate a plurality of importance scores relative to the previous symptom queries respectively according to the amount variation.

19. A non-transitory computer-readable storage medium, storing at least one instruction program executed by a processor to perform a control method, the control method comprising:
receiving an input state;
utilizing a neural network model to select a current action, from a plurality of candidate symptom queries and a plurality of candidate disease predictions, according to the input state;
in response to the current action is a first symptom query, generating a diagnostic tree for simulating a plurality of possible diagnosis paths, each of the possible diagnosis paths passing through the first symptom query and terminated at respective one of the candidate disease predictions, the possible diagnosis paths involving a positive hypothesis of the first symptom query and a negative hypothesis of the first symptom query; and
generating a symptom query interpretation about the first symptom query according to the diagnostic tree.

20. The non-transitory computer-readable storage medium as claimed in claim 19, wherein the control method further comprises:
in response to the current action is a disease prediction of a complete diagnose procedure, generating a post hoc diagnostic tree comprising all possible diagnosis paths diverged at the previous symptom queries;
calculating an amount variation of the disease hypotheses considered by the neural network model before and after each of the previous symptom queries according to the post hoc diagnostic tree; and
calculating a plurality of importance scores relative to the previous symptom queries respectively according to the amount variation.
